SEQUENCE LISTING (11 sequences)

SEQ ID NO: 1 - protein, 5 aa, synthetic construct
DYWMQ

SEQ ID NO: 2 - protein, 17 aa, synthetic construct
TIYPGDGDTGYAQKFQG

SEQ ID NO: 3 - protein, 11 aa, synthetic construct
GDYYGSNSLDY

SEQ ID NO: 4 - protein, 11 aa, synthetic construct
KASQDVSTVVA

SEQ ID NO: 5 - protein, 7 aa, synthetic construct
SASYRYI

SEQ ID NO: 6 - protein, 9 aa, synthetic construct
QQHYSPPYT

SEQ ID NO: 7 - protein, 120 aa, synthetic construct [sequence omitted]

SEQ ID NO: 8 - protein, 108 aa, synthetic construct
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKR

SEQ ID NO: 9 - protein, 108 aa, synthetic construct
DIVMAQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKR

SEQ ID NO: 10 - protein, 449 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 214 aa, synthetic construct
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0177519 A1
(43) Pub. Date: Jun. 5, 2025

(54) USE OF ISATUXIMAB IN COMBINATION WITH OTHER AGENTS FOR THE TREATMENT OF MULTIPLE MYELOMA

(71) Applicant: Sanofi-Aventis U.S. LLC, Bridgewater, NJ (US)

(72) Inventors: Marielle CHIRON BLONDEL, Paris (FR); Alexandre DESJONQUERES, Chilly-Mazarin (FR); Zandra Karina KLIPPEL, Rueil Malmaison (FR); Marco MELONI, Montrouge (FR); Angela VIRONE-ODDOS, Clamart (FR)

(21) Appl. No.: 18/844,343

(22) PCT Filed: Mar. 7, 2023
(86) PCT No.: PCT/US2023/063869

§ 371 Date: Sep. 5, 2024

Related U.S. Application Data
(60) Provisional application No. 63/317,253, filed on Mar. 7, 2022.

(51) Int. Cl. A61K 39/395; A61K 35/17; A61K 38/20; A61K 39/00; C07K 16/28

(52) U.S. Cl. CPC A61K 39/3955; A61K 35/17; A61K 38/2013; C07K 16/2896; A61K 2039/545

(57) ABSTRACT

The present disclosure provides methods for treating multiple myeloma comprising administering an anti-CD38 antibody and an Interleukin-2 analog to an individual in need thereof and optionally administering Natural Killer (NK) cells having expression of CD38 reduced or knocked-out.

### CROSS REFERENCE TO RELATED APPLICATIONS

This application claims the priority benefit of U.S. Provisional Application No. 63/317,253, filed Mar. 7, 2022, the contents of which are incorporated herein by reference in their entirety.

### REFERENCE TO AN ELECTRONIC SEQUENCE LISTING

The contents of the electronic sequence listing (183952034340SEQLIST.xml; Size: 11,571 bytes; and Date of Creation: Mar. 6, 2023) is herein incorporated by reference in its entirety.

### FIELD

The present disclosure relates to methods of treating multiple myeloma by administering an anti-CD38 antibody, e.g., isatuximab.

### BACKGROUND

Multiple myeloma (MM) is a malignant plasma cell disease that is characterized by clonal proliferation of plasma cells in the bone marrow (BM) and the production of excessive amounts of a monoclonal immunoglobulin (usually of the IgG or IgA type or free urinary light chain, i.e., paraprotein, M-protein or M-component). Patients with MM can experience bone pain, bone fractures, fatigue, anemia, infections, hypercalcemia, and kidney problems (Rollig et al. (2015) Lancet. 385(9983):2197-208). The expression of CD38 is especially notable in MM as >98% of patients are positive for this protein (Goldmacher et al. (1994) Blood. 84(9):3017-25; Lin et al. (2004) Am J Clin Pathol. 121(4):482-8). The strong and uniform expression of CD38 on malignant clonal MM cells contrasts with the restricted expression pattern on normal cells suggesting this antigen may be useful for specific targeting of tumor cells.

In general, MM patients will receive treatment regimens during their lifespan that include such agents such as proteasome inhibitors (e.g., bortezomib, ixazomib, and carfilzomib) and immune modulatory agents or “IMiDs®” (e.g., lenalidomide, pomalidomide, and thalidomide), monoclonal antibodies (e.g., elotuzumab), histone deacetylase (HDAC) inhibitors (e.g., panobinostat) alone or in combination.

Despite significant advances and prolongation in overall survival (OS), multiple myeloma (MM) remains incurable, with the majority of patients relapsing and requiring additional treatment (Kumar S K, Rajkumar V, Kyle R A, et al. Multiple myeloma. Nat Rev Dis Primer. 2017; 3: 17046).

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

Provided herein are anti-CD38 antibodies and an Interleukin-2 analogs (IL-2 analogs) for use in treating multiple myeloma. In some embodiments, the anti-CD38 antibodies and IL-2 analogs provided herein are combined with NK cells have had expression of CD38 reduced or knocked-out (NK-CD38KO cells).

Provided herein are methods of treating multiple myeloma comprising administering an anti-CD38 antibody and an Interleukin-2 analog (IL-2 analog) to an individual, wherein the anti-CD38 antibody is administered on Days 1, 8, 15, and 22 of a first 28-day cycle and then administered on Days 1 and 15 of a 28-day cycle for at least one additional cycle and administering the IL-2 analog to the individual, wherein the IL-2 analog is administered once every two weeks or once every three weeks. In some embodiments, the method comprises administering NK-CD38KO cells in combination with an anti-CD38 antibody and/or an IL-2 analog.

Provided herein are uses of an anti-CD38 antibody for the treatment of multiple myeloma, wherein the anti-CD38 antibody is administered to an individual in combination with an Interleukin-2 analog (IL-2 analog), wherein the anti-CD38 antibody is administered on Days 1, 8, 15, and 22 of a first 28-day cycle and then administered on Days 1 and 15 of a 28-day cycle for at least one additional cycle and wherein the IL-2 analog is administered once every two weeks or once every three weeks. In some embodiments, the anti-CD38 antibody and IL-2 analog are administered in combination with cells NK-CD38KO cells.

### BRIEF DESCRIPTION OF THE DRAWINGS

FIG. 1 is a bar graph showing fratricide effect is avoided with CD38 KO K-NK cells from BC50 sample compared to wild type KNK cells in the presence of isatuximab (light grey) or isotype control (dark grey).

FIG. 2 is a bar graph showing fratricide effect is avoided with CD38 KO K-NK cells from BC45 sample compared to wild type KNK cells in the presence of isatuximab (light grey) or isotype control (dark grey).

### DETAILED DESCRIPTION

### Overview

This disclosure provides anti-CD38 antibodies and an Interleukin-2 analogs (IL-2 analogs) for use in treating multiple myeloma. In some embodiments, the anti-CD38 antibodies and IL-2 analogs provided herein are combined with NK cells have had expression of CD38 reduced or knocked-out (NK-CD38KO cells). Provided herein are methods for treating or delaying the progression of multiple myeloma (MM) in an individual. In some embodiments, the patient has not previously received treatment for MM (e.g., Newly Diagnosed MM). In some embodiments, the individual has received one, two, three, or more than three prior therapies for MM. In some embodiments, the individual has received one or more prior lines of therapy including an anti-CD38 medication and an anti-BCMA medication.

Provided herein are methods of treating multiple myeloma comprising administering an anti-CD38 antibody and an Interleukin-2 analog (IL-2 analog) to an individual. Provided herein are uses of an anti-CD38 antibody for the treatment of multiple myeloma, wherein the anti-CD38 antibody is administered to an individual in combination with an IL-2 analog. In some embodiments, of the methods and uses provided herein, the anti-CD38 antibody and IL-2 analog are administered in combination with cells NK-CD38KO cells.

The methods comprise administering to the individual an effective amount of an anti-CD38 antibody (e.g., isatuximab), in combination with Natural Killer (NK) cells that have had CD38 expression “knocked-out” (e.g., the cells have genetically modified to remove all or part of the CD38 gene such that CD38 is not expressed by the cell), or IL-15 or analog thereof, or a combination thereof. In some embodiments dexamethasone is also administered. In some embodiments, the treatment extends the progression free survival (PFS) and/or the overall survival (OS) of the individual. In some embodiments, the treatment extends the progression free survival (PFS) and/or the overall survival (OS) of the individual, as compared to an individual who is not receiving treatment. In some embodiments, the individual is negative for minimal residual disease (MRD) (e.g., at a threshold of 10⁻⁴ or less, 10⁻⁵ or less, or 10⁻⁶ or less) after treatment (also referred to as “MRD negative”).

### Anti-CD38 Antibodies

In some embodiments, the anti-CD38 antibody binds to human CD38. In some embodiments, the anti-CD38 antibody is a human antibody, a humanized antibody, or a chimeric antibody. In some embodiments, the anti-CD38 antibody comprises (a) a heavy chain variable domain (VH) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6). In some embodiments, the anti-CD38 antibody comprises a heavy chain variable domain (VH) that comprises an amino acid sequence that is at least 90% identical (e.g., at least any one of 91%, 92%, 94%, 95%, 96%, 97%, 98%, or 99%, including any range between these values) to SEQ ID NO: 7. Additionally or alternatively, in some embodiments, the anti-CD38 antibody comprises a light chain variable domain (V_{L}) that comprises an amino acid sequence that is at least 90% identical (e.g., at least any one of 91%, 92%, 94%, 95%, 96%, 97%, 98%, or 99%, including any range between these values) to SEQ ID NO: 8 or SEQ ID NO: 9. In some embodiments, the anti-CD38 antibody comprises a VH that comprises SEQ ID NO: 7 and a V_{L} that comprises SEQ ID NO: 8 or SEQ ID NO: 9.

(SEQ ID NO: 7)
QVQLVQSGAE VAKPGTSVKL SCKASGYTFT DYWMQWVKQR
PGQGLEWIGT IYPGDGDTGY AQKFQGKATL TADKSSKTVY
MHLSSLASED SAVYYCARGD YYGSNSLDYW GQGTSVTVSS
(SEQ ID NO: 8)
DIVMTQSHLS MSTSLGDPVS ITCKASQDVS TVVAWYQQKP
GQSPRRLIYS ASYRYIGVPD RFTGSGSGTD FTFTISSVQA
EDLAVYYCQQ HYSPPYTFGG GTKLEIKR
(SEQ ID NO: 9)
DIVMAQSHLS MSTSLGDPVS ITCKASQDVS TVVAWYQQKP
GQSPRRLIYS ASYRYIGVPD RFTGSGSGTD FTFTISSVQA
EDLAVYYCQQ HYSPPYTFGG GTKLEIKR

In some embodiments, the anti-CD38 antibody is isatuximab (CAS Registry Number: 1461640-62-9). Isatuximab, also known as hu38SB19 and SAR650984, is an anti-CD38 antibody described in WO 2008/047242 and U.S. Pat. No. 8,153,765, the contents of both of which are incorporated by reference herein in their entirety.

The heavy chain of isatuximab comprises the amino acid sequence:

(SEQ ID NO: 10)
QVQLVQSGAE VAKPGTSVKL SCKASGYTFT DYWMQWVKQR
PGQGLEWIGT IYPGDGDTGY AQKFQGKATL TADKSSKTVY
MHLSSLASED SAVYYCARGD YYGSNSLDYW GQGTSVTVSS
ASKTGPSVFP LAPSSKSTSG GTAALGCLVK DYFEPEVTVS
WNSGALTSGV HTFPAVLQSS GLYSLSSVVT VPSSSLGTQT
YICNVNHKPS NTKVDKKVEP KSCDKTHTCP PCPAPELLGG
PSVFLFPPKP KDTLMISRTP EVTCVVVDVS HEDPEVKFNW
YVDGVEVHNA KTKPREEQYN STYRVVSVLT VLHQDWLNGK
EYKCKVSNKA LPAPIEKTIS KAKGQPREPQ VYTLPPSRDE
LTKNQVSLTC LVKGFYPSDI AVEWESNGQP ENNYKTTPPV
LDSDGSFFLY SKLTVDKSRW QQGNVFSCSV MHEALHNHYT
QKSLSLSPG

and the light chain of isatuximab comprises the amino acid sequence:

(SEQ ID NO: 11)
DIVMTQSHLS MSTSLGDPVS ITCKASQDVS TVVAWYQQKP
GQSPRRLIYS ASYRYIGVPD RFTGSGSGTD FTFTISSVQA
EDLAVYYCQQ HYSPPYTFGG GTKLEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

The anti-CD38 antibodies may be produced using recombinant methods. For recombinant production of an anti-antigen antibody, nucleic acid encoding the antibody is isolated and inserted into a replicable vector for further cloning (amplification of the DNA) or for expression. DNA encoding the antibody may be readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence. The vector is typically transformed into a host cell suitable for expression of the nucleic acid. In some embodiments, the host cell is a eukaryotic cell or a prokaryotic cell. In some embodiments, the eukaryotic host cell is a mammalian cell. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as NSO and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J., 2003), pp. 255-268. The anti-CD38 antibody prepared from the cells can be purified using, for example, hydroxylapatite chromatography, hydrophobic interaction chromatography, gel electrophoresis, dialysis, and affinity chromatography, with affinity chromatography being among one of the typically preferred purification steps. In general, various methodologies for preparing antibodies for use in research, testing, and clinical applications are well-established in the art, consistent with the above-described methodologies and/or as deemed appropriate by one skilled in the art.

In some embodiments the IL-2 analog is an IL-2 that has been pegylated. In some embodiments, the IL-2 analog has (such as includes or comprises) a non-natural amino acid such as N6-((2-azidoethoxy)-carbonyl)-L-lysine (AzK). In some embodiments, the non-natural amino acid is singly pegylated with a linear PEG group having an average molecular weight of 30 kDa. Suitable IL-2 analogs are described, for example, in WO2020/163532 which in incorporated herein it its entirety.

In some embodiments, Natural Killer (NK) cells modified such that expression of CD38 reduced or knocked-out (NK-CD38KO cells) are used in conjunction with isatuximab and/or an IL-2 analog for the treatment of multiple myeloma. Such NK-CD38KO cells can be produced, for example, by a method described in WO2021087466, the teachings of which are incorporated in their entirety.

In some embodiments, the individual received one or more lines of therapy for the treatment of multiple myeloma prior to receiving the first cycle of treatment with anti-CD38 antibody, IL-2 analog and optionally NK-CD38KO cells. The anti-CD38 medication includes, for example, mono or multi specific binding agents that are capable of specifically binding human CD38. The anti-BCMA medication includes, for example, mono or multi specific binding agents that are capable of specifically binding human BCMA. The binding agents can be, for example, monoclonal antibodies, di- or tri-specific antibodies, or antibody analogs that contain antigen specific regions of a traditional antibody that can specifically bind CD38 or BCMA, respectively. The binding agent can also be an antibody-drug conjugate.

### Pharmaceutical Compositions and Formulations

Also provided herein are pharmaceutical compositions and formulations, e.g., for the treatment of multiple myeloma comprising an anti-CD38 antibody (such as isatuximab), IL-2 analog, NK-CD38KO cells, and/or dexamethasone. In some embodiments, each of the anti-CD38 antibody, the IL-2 analog, the NK-CD38KO cells and the optional dexamethasone is provided as a separate pharmaceutical composition. In some embodiments, the pharmaceutical compositions and formulations further comprise a pharmaceutically acceptable carrier.

In some embodiments, the anti-CD38 antibody is in a formulation suitable for intravenous administration, for example comprising about 20 mg/mL (500 mg/25 mL) antibody, about 20 mM histidine, about 10% (w/v) sucrose, about 0.02% (w/v) polysorbate 80 at pH 6.0. In some embodiments, the anti-CD38 antibody is in a formulation comprising about 20 mg/mL antibody, about 100 mg/mL sucrose, 2.22 mg/mL histidine hydrochloride monohydrate, about 1.46 mg/ml histidine, and about 0.2 mg/ml polysorbate 80. In some embodiments, the formulation comprises water for injection (WFI), such as sterile water for injection (SWFI). In some embodiments, the formulation is sterile. In some embodiments, a single use of the formulation comprises 5 ml of the formulation (i.e., 100 mg anti-CD38 antibody). In some embodiments, the single use 5 ml formulation is provided in, e.g., a type 16 mL colorless clear glass vial fitted with elastomeric closure. In some embodiments, the fill volume of the vial has been established to ensure removal of 5 mL. In some embodiments, the fill volume is 5.4 mL. In some embodiments, a single use of the formulation comprises 25 ml of the formulation (i.e., 500 mg anti-CD38 antibody). In some embodiments, the single use 25 ml formulation is provided in, e.g., a 30 mL colorless clear glass vial fitted with elastomeric closure. In some embodiments, the fill volume of the vial has been established to ensure removal of 25 mL. In some embodiments, the formulation is stable for at least about 6, 12, 18, 24, 30, or 36 months, including any range in between these values, at a temperature between about 2° C. and about 8° C. and protected from light. In some embodiments, the formulation is diluted for infusion in 0.9% sodium chloride or 5% dextrose. In some embodiments, the diluted infusion solution is stable for up to about 6, 12, 18, 24, 30, 36, 42, or 48 hours, including any range in between these values, between about 2° C. and about 8° C. In some embodiments, the diluted solution for infusion is stable following storage between about 2° C. and about 8° C. for a further 8 hours (including the infusion time) at room temperature. In some embodiments, the diluted solution for infusion is stable in the presence of light. In some embodiments the bag in which the diluted solution for infusion is stored is fabricated from polyolefins (PO), polyethylene (PE), polypropylene (PP), polyvinyl chloride (PVC) with di(ethylhexyl)phthalate (DEHP) or ethyl vinyl acetate (EVA). In some embodiments, the tubing used for infusion is fabricated from PE, PVC (with or without DEHP), polybutyldiene (PBD), or polyurethane (PU) with an in-line filter (polyethersulfone (PES), polysulfone or nylon).

In some embodiments, the anti-CD38 antibody is provided as formulation for subcutaneous administration. In some embodiments the anti-C38 antibody comprises isatuximab at a concentration of 140 mg/mL, 9 mM histidine, 110 mM Arg-Cl, 2% (w/v) sucrose, and 0.4% (w/v) Poloxamer 188.

Use of Anti-CD38 Antibody in Combination with other Agents for the Treatment of Multiple Myeloma

Provided herein are anti-CD38 antibodies for use in combination with one or more of an IL-2 analog, Natural Killer (NK) cells modified such that expression of CD38 reduced or knocked-out (NK-CD38KO cells), and dexamethasone for the treatment of multiple myeloma in an individual (e.g., a human individual). In some embodiments, the use comprises administering to the individual an effective amount of an anti-CD38 antibody (e.g., an anti-CD38 antibody comprising (a) a heavy chain variable domain (V_{H}) that comprises: a CDR-H1 comprising the amino acid sequence DYWMQ (SEQ ID NO: 1), a CDR-H2 comprising the amino acid sequence TIYPGDGDTGYAQKFQG (SEQ ID NO: 2), and a CDR-H3 comprising the amino acid sequence GDYYGSNSLDY (SEQ ID NO: 3), and (b) a light chain variable domain (V_{L}) that comprises: a CDR-L1 comprising the amino acid sequence KASQDVSTVVA (SEQ ID NO: 4), a CDR-L2 comprising the amino acid sequence SASYRYI (SEQ ID NO: 5), and a CDR-L3 comprising the amino acid sequence QQHYSPPYT (SEQ ID NO: 6). In some embodiments, the anti-CD38 antibody is isatuximab.

In some embodiments, the method comprises administering the anti-CD38 antibody (e.g., isatuximab) to the individual at a dose of 10 mg/kg on Days 1, 8, 15, and 22 of a first 28-day cycle and administering the anti-CD38 antibody at a dose of 10 mg/kg on Days 1 and 15 of a 28-day cycle for at least one additional cycle.

In some embodiments, the method comprises administering the anti-CD38 antibody (e.g., isatuximab) at a dose of 10 mg/kg once every 28 days of one or more additional 28-day cycles following at least 11 cycles of administering the anti-CD38 antibody at a dose of 10 mg/kg on Days 1 and 15 of a 28-day cycle.

In some embodiments, the method comprises administering the anti-CD38 antibody (e.g., isatuximab) at a dose of 10 mg/kg once every 28 days of one or more additional 28-day cycles after the subject has achieved at least Very Good Partial Response (VGPR) while being treated with the anti-CD38 antibody.

In some embodiments, the method comprises administering the anti-CD38 antibody (e.g., isatuximab) at a dose of 10 mg/kg once every 28 days of one or more additional 28-day cycles after the subject has achieved MRD negativity while being treated with the anti-CD38 antibody.

In some embodiments, the method comprises administering the anti-CD38 antibody (e.g., isatuximab) at a dose of 10 mg/kg once every 28 days of one or more additional 28-day cycles after the subject has achieved MRD negativity while being treated with the anti-CD38 antibody.

In some embodiments described herein, the IL-2 analog is IL-2 modified to include a non-natural amino acid, N6-((2-azidoethoxy)-carbonyl)-L-lysine (AzK), and wherein the AzK is pegylated. In some embodiments, the IL-2 analog is administered once every two weeks, or once every three weeks, or combination thereof. In some embodiments, the IL-2 analog is administered at a dose of 24 ug/kg. In some embodiments, the IL-2 analog is administered at a dose of 32 ug/kg. In some embodiments, the IL-2 analog is administered at a dose of 16 ug/kg.

In some embodiments, the treatment regimens described herein extends the progression-free survival (PFS) of the individual.

In some embodiments, the multiple myeloma is smoldering multiple myeloma (SMM). In some embodiments, the multiple myeloma is newly diagnosed multiple myeloma. In some embodiments, the multiple myeloma is relapsed and/or refractory multiple myeloma (RRMM). In some embodiments, the individual received 1, 2, or 3 prior therapies for multiple myeloma. In some embodiments, the individual received more than three prior therapies with multiple myeloma. In some embodiments, the individual received prior therapy with a proteasome inhibitor. In some embodiments, the individual received prior therapy with an immunomodulatory agent. In some embodiments, the individual has not received prior treatment with an anti-CD38 antibody.

In some embodiments, the individual has received prior treatment with an anti-CD38 antibody. In some embodiments, the prior anti-CD38 antibody was daratumumab. In some embodiments, the prior anti-CD38 antibody was isatuximab.

### Articles of Manufacture or Kits

In another embodiment of the invention, an article of manufacture or a kit is provided comprising an anti-CD38 antibody (such as isatuximab). In some embodiments, the article of manufacture or kit further comprises at least one additional agent (e.g., one or more of IL-2 analog, NK-CD38KO cells, or dexamethasone). In some embodiments, the article of manufacture or kit further comprises package insert comprising instructions for using the anti-CD38 antibody (e.g., isatuximab) and other agents according to a use described herein to treat or delay progression of multiple myeloma (e.g., smoldering multiple myeloma, newly diagnosed multiple myeloma, refractory multiple myeloma, or relapsed and refractory multiple myeloma).

### Definitions

As used in this specification and the appended claims, the singular forms “a,” “an” and “the” include plural referents unless the content clearly dictates otherwise. Thus, for example, reference to “a molecule” optionally includes a combination of two or more such molecules, and the like.

“Sustained response” refers to the sustained effect on preventing or delaying progression of a disease (e.g., multiple myeloma) and/or improving one or more response criteria after cessation of a treatment. For example, response to treatment for multiple myeloma may be measured according to the criteria in Kumar et al. (2016) “International Myeloma Working Group consensus criteria for response and minimal residual disease assessment in multiple myeloma.” Lancet Oncol. 17(8): e328-e346) and Durie et al. (2006) “International uniform response criteria for multiple myeloma. Leukemia. 20: 1467-1473. (See also Table A below.) In some embodiments, the sustained response has a duration at least the same as the treatment duration, at least 1.5×, 2.0×, 2.5×, or 3.0× length of the treatment duration.

TABLE A
Standard International Myeloma Working Group (IMWG) Response Criteria

| Response | IMWG criteria |
|---|---|
| Complete | Negative immunofixation on the serum and urine and |
| Response | disappearance of any soft tissue plasmacytomas and |
| (CR) | <5% plasma cells in bone marrow aspirates. |
| | Two consecutive assessments are needed. No known evidence of |
| | progressive disease or new bone lesions if radiographic studies were |
| | performed |
| Stringent | CR as defined above plus: |
| Complete | Normal free light chain ratio (0.26 to 1.65) and |
| Response | Absence of clonal cells in bone marrow by immunohistochemistry (κ/λ |
| (sCR) | ratio ≤4:1 or ≥1:2 for κ and λ patients, respectively, after counting ≥100 |
| | plasma cells). |
| | Two consecutive assessments of laboratory parameters are needed. No |
| | known evidence of progressive disease or new bone lesions if |
| | radiographic studies were performed |
| Very Good | Serum and urine M protein detectable by immunofixation but not on |
| Partial | electrophoresis or |
| Response | ≥90% reduction in serum M protein plus urine M protein level <100 mg/24 |
| (VGPR) | hour. |
| | ≥90% decrease in the sum of the products of maximal perpendicular |
| | diameter (SPD) compared to baseline in soft tissue plasmacytoma. |
| | Two consecutive assessments are needed. No known evidence of |
| | progressive disease or new bone lesions if radiographic studies were |
| | performed. |
| Partial | ≥50% reduction of serum M protein and reduction in 24 hours urinary M |
| Response | protein by ≥90% or to <200 mg/24 hour |
| (PR) | In addition to the above listed criteria, if present at baseline, a ≥50% |
| | reduction in the size (sum of the products of the maximal perpendicular |
| | diameters or “SPD”) of soft tissue plasmacytomas is also required |
| | Two consecutive assessments are needed. No known evidence of |
| | progressive disease or new bone lesions if radiographic studies were |
| | performed. |
| Minimal | ≥25% but ≤49% reduction in serum M protein and reduction in 24 h urine M |
| Response | protein by 50 to 89%, which still exceed 200 mg/24 hour. |
| (MR) | In addition to the above listed criteria, if present at baseline, ≥50% reduction |
| | in size (SPD) of soft tissue plasmacytomas is also required. |
| | Two consecutive assessments are needed. No known evidence of |
| | progressive disease or new bone lesions if radiographic studies were |
| | performed. |
| Stable | Not meeting criteria for CR, VGPR, PR, MR, or progressive disease. |
| Disease | No known evidence of progressive disease or new bone lesions if |
| (SD) | radiographic studies were performed. |
| Progressive | Any 1 or more of the following criteria: |
| disease | Increase of ≥25% from lowest confirmed value in any 1 of the following |
| (PD) | criteria: |
| | Serum M protein (the absolute increase must be ≥0.5 g/dL). |
| | Serum M protein increase ≥1 g/dL if the lowest M component was ≥5 g/dL. |
| | Urine M-component (the absolute increase must be ≥200 mg/24 hour). |
| | Appearance of new lesion(s), ≥50% increase from nadir in SPD of >1 lesion, |
| | or ≥50% increase in the longest diameter of a previous lesion >1 cm in short |
| | axis. |
| | Two consecutive assessments are needed for PD on M protein. |

‡SPD, sum of the products of the maximal perpendicular diameters of measured lesions

Methods of measuring serum and urine M-protein levels are well known in the art and described in, e.g., Jenkins (2009) Clin Biochem Rev. 30(3): 119-122; Leung, Nelson “Chapter 8: Clinical Tests for Monoclonal Proteins.” Onco-Nephrology Curriculum, American Society of Nephrology 2016, pages 1-5).

In some embodiments, VGPR is assessed according to the criteria in Kumar et al. (2016) “International Myeloma Working Group consensus criteria for response and minimal residual disease assessment in multiple myeloma.” Lancet Oncol. 17(8): e328-e346) and Durie et al. (2006) “International uniform response criteria for multiple myeloma. Leukemia. 20: 1467-1473, the contents of which are incorporated herein by reference in their entireties. (See also Table A.)

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those that can reasonably be administered to a subject mammal to provide an effective dose of the active ingredient employed.

As used herein, the term “treatment” refers to clinical intervention designed to alter the natural course of the disease or cell (e.g., cancer cell) being treated during clinical pathology. Desirable effects of treatment include decreasing the rate of disease progression, ameliorating, or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late-stage cancer, such as development of metastasis, may be delayed.

An “effective amount” is at least the minimum amount required to effect a measurable improvement or prevention of a particular disorder. An effective amount herein may vary according to factors such as the disease state, age, sex, and weight of the individual/patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality. As such, “in conjunction with” refers to administration of one treatment modality before, during, or after administration of the other treatment modality to the individual.

A “subject” or an “individual” for purposes of treatment refers to any animal classified as a mammal, including humans, domestic and farm animals, and zoo, sports, or pet animals, such as dogs, horses, cats, cows, etc. Preferably, the mammal is human.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

Human light chains are typically classified as kappa and lambda light chains, and human heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, the variable and constant domains typically are joined by a “J” region of about 12 or more amino acids, with the heavy chain also including a “D” region of about 10 more amino acids. See, e.g., FUNDAMENTAL IMMUNOLOGY (Paul, W., ed., Raven Press, 2nd ed., 1989), which is incorporated by reference in its entirety for all purposes. The variable regions of each light/heavy chain pair typically form an antigen binding site. The variable domains of antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise, in order, the domains FR1, CDR1, FR2, CDR2, FR3, CDR3, and FR4.

The term “CDR set” refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs.

The term “Fc” as used herein refers to the sequence of a non-antigen-binding fragment that would result from digestion of an antibody or produced by other means, whether in monomeric or multimeric form, and can contain the hinge region. The original immunoglobulin source of the native Fc is preferably of human origin and can be any of the immunoglobulins. Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, IgGA2, and IgG4). One example of a Fc is a disulfide-bonded dimer resulting from papain digestion of an IgG. The term “native Fc” as used herein is generic to the monomeric, dimeric, and multimeric forms.

As used herein, the term “overall response rate” or “ORR” refers to the proportion of individuals/patients with stringent complete response (sCR), complete response (CR), very good partial response (VGPR), and partial response (PR), as assessed by the IRC using the IMWG response criteria described in Kumar et al. (2016) “International Myeloma Working Group consensus criteria for response and minimal residual disease assessment in multiple myeloma.” Lancet Oncol. 17(8): e328-e346 and Durie et al. (2006) “International uniform response criteria for multiple myeloma. Leukemia. 20: 1467-1473. See also Table A herein.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The present disclosure will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1

### Measurements of Cytotoxic Activities In Vitro

### Antibody Dependent Cellular Cytotoxicity (ADCC)

Antibody dependent cellular cytotoxicity (ADCC) against LP-1 RFP multiple myeloma cells (target cells) using WT or CD38KO K-NK cells (effector cells) in combination with isatuximab and SAR444245 (pegylated IL-2 analog) was measured over time by Incucyte® live-cell imaging and analysis system (Essen Bioscience).

LP-1 RFP cells were generated by infecting LP-1 cells (DSMZ) with Incucyte® Nuclight Red Lentivirus (Sartorius) to express red fluorescent protein (RFP). LP-1 RFP cells were maintained in IMDM medium (Gibco, #12440053) supplemented with 20% fetal bovine serum (FBS heat inactivated, Biowest, #S181H-100), 1% L-Glutamine (Gibco, #25030-024), and incubated at 37° C. with 5% C02. LP-1 RFP cells were centrifuged for 5 minutes at 300 g, resuspended in RPMI1640 complete medium (RPMI1640 supplemented with 10% fetal bovine serum—FBS, Biowest, #S181H-100-, 1% L-Glutamine-Gibco, #25030-024) before being added to Incucyte® plates (details below).

Natural Killer (NK) cells having expression of CD38 reduced or knocked-out (CD38KO K-NK cells) were generated using peripheral blood NK cells isolated from two healthy donors (BC45 and BC50) and expanded using PM21 particle technology to produce highly activated K-NK cells. BC45 cells have the VN phenotype for CD16 while BC50 cells have the F/V phenotype.

To produce CD38KO K-NK cells, CRISPR gene editing was applied during NK cell expansion by electroporating with Cas9/RNP complexes targeting CD38. After expansion, WT and CD38KO K-NK cells were frozen and kept at −150° C. WT and CD38KO K-NK cells were thawed, seeded in RPMI1640 complete medium supplemented with 50 U/mL of IL-2 (Peprotech, #200-02), and incubated at 37° C. with 5% CO2 for 16-20 hours. Cells were then centrifuged for 5 minutes at 300 g before being added to Incucyte® plates.

Compounds and cells were added to the appropriate wells of an Incucyte® plate (Poly-D lysine treated 96-well flat bottom microplate CellCoat™, Greiner Bio-One; #655946) in the following order (final volume of each well: 200 μl):

Isatuximab (or isotype control) (previously diluted in RPMI1640 complete medium) was added to the appropriate wells to have a final concentration of either 0.1, 1 or 10 ng/ml (50 μl/well).

SAR444245 (previously diluted in RPMI1640 complete medium) was then added to the appropriate wells to have a final concentration of 333.33 ng/ml (50p/well).

LP-1 RFP cells (target cells, T) were then added to each well (to have 20000 LP1-RFP cells/well) (50 μl/well).

WT or CD38KO K-NK cells (effector cells, E) were then added to the appropriate wells. Either 20000, 60000 or 100000 cells were added to evaluate different E:T ratios (1:1, 3:1 and 5:1, respectively) (50 μl/well).

The Incucyte® plate was then centrifuged for 1 minute at 100 g before being placed into an Incucyte® (IncucyteS3, EssenBio) which was housed within a dedicated incubator at 37° C. with 5% CO2. Images (4 images/well) were taken every 2 hours with a 10× objective and a standard scan type using the phase and red channels. The growth of LP-1 RFP target cells was monitored by fluorescent imaging up to 90 hours, and the number of live target cells quantified using the IncucyteS3 software and normalized to the number of live target cells time zero.

Experiments were performed at least 2 times with WT and CD38KO K-NK cells from two donors (BC45 and BC50) with each condition in duplicate.

### Fratricide Effect

WT and CD38KO K-NK cells were thawed, seeded in RPMI1640 complete medium (RPMI1640 supplemented with 10% fetal bovine serum—FBS, Biowest, #S181H-100-, 1% L-Glutamine-Gibco, #25030-024) additionally supplemented with 50 U/mL of IL-2 (Peprotech, #200-02), and incubated at 37° C. with 5% C02 for 16-20 hours. Cells were then centrifuged for 5 minutes at 300 g, counted, and resuspended in RPMI1640 complete medium to have 50000 K-NK cells/50 μl.

Compounds and cells were added to the appropriate wells of a Corning™ 96-Well Clear Ultra Low Attachment Microplate (Corning, #7007) in the following order (final volume of each well: 200 μl):

RPMI1640 complete medium was added to each well (100 μl/well).

Isatuximab (or isotype control) (previously diluted in RPMI1640 complete medium) was added to the appropriate wells to have a final concentration of 10 ng/ml (501/well).

WT or CD38KO K-NK cells were then added to the appropriate wells (to have 50000 K-NK cells/well) (50 μl/well).

The plate was then centrifuged for 1 minute at 100 g before being placed into an incubator at 37° C. with 5% C02 for 4 hours.

To quantify fratricide, WT and CD38KO K-NK cells were stained with either the Annexin V-FITC Kit or with DiOC6/DRAQ7.

For labelling with Annexin V-FITC kit (Miltenyi Biotec; #130-092-052):

After 4 hours incubation, the plate was centrifuged for 5 minutes at 300 g, the medium removed, and the pellet resuspended in 200 μL of Annexin V-FITC kit buffer solution. The plate was centrifuged again for 5 minutes at 300 g.

The buffer was removed, then the pellet was resuspended in 100 μL of Annexin V-FITC kit buffer solution and 5 μL of Annexin V-FITC were added to the appropriate wells.

The plate was incubated at room temperature and in the dark for 15 minutes.

100 μL of Annexin V-FITC kit buffer solution was added, and the plate was centrifuged for 5 minutes at 300 g, and the buffer removed.

The pellet was resuspended in 200 μL of Annexin V-FITC kit buffer solution, and the plate was centrifuged again for 5 minutes at 300 g.

The buffer was removed, and the pellet resuspended in 100 μL of Annexin V-FITC kit buffer solution.

1 L of propidium iodide (PI) was added to the appropriate wells just before the analysis.

Samples were then analyzed with the MACSQuant 16 flow cytometer (Miltenyi Biotec).

Data were Analyzed with VenturiOne Software Using the Following Strategy:

The K-NK cell population excluding debris was gated, followed by gating for single cells. From single cells, viable PIneg and Annexin V-FITCneg cells were quantified.

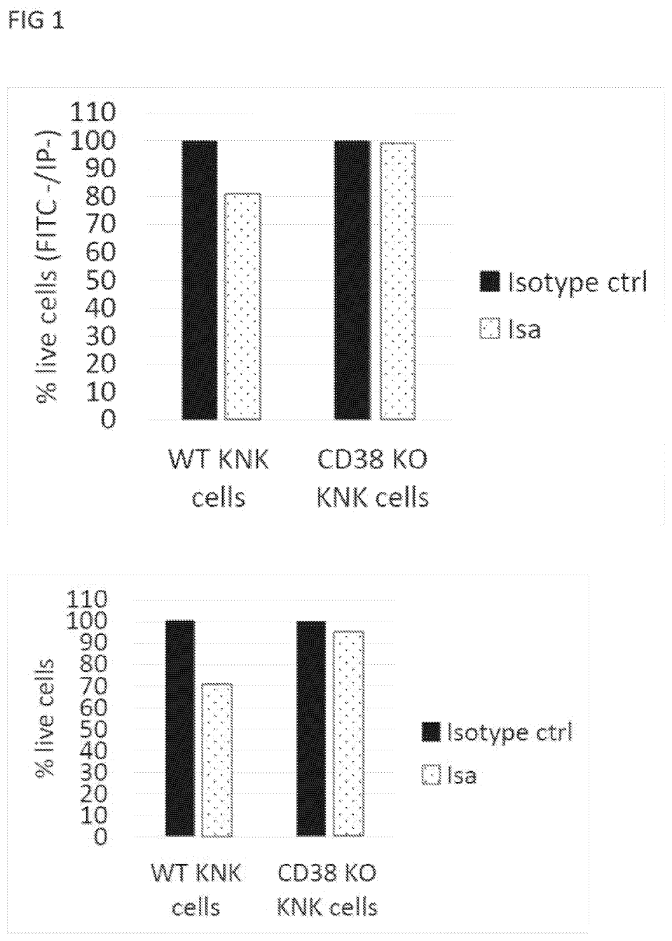

For the graphic representation, viable K-NK cells (Annexin Vneg/PIneg) were quantified as percentage of viable cells in the presence of isatuximab normalized to the percentage of viable cells in the presence of isotype control (100%) (see FIG. 1).

For labelling with DiOC6/DRAQ7:

After 4 hours incubation, 20 μL of 100 nM DiOC6 (3,3′-Dihexyloxacarbocyanine Iodide, ThermoFisher, #D273) were added to the appropriate wells.

The plate was incubated at 37° C. with 5% C02 for 20 min.

The plate was then centrifuged for 5 min at 300 g, the medium removed, and the pellet was resuspended in 100 μL of 3 μM DRAQ7 (BD Biosciences, #564904).

Samples were then analyzed with the MACSQuant 16 flow cytometer (Miltenyi Biotec).

Data were Analyzed with VenturiOne Software Using the Following Strategy:

The K-NK cell population excluding debris was gated, followed by gating for single cells. From single cells, viable DiOC6pos and DRAQ7neg cells were quantified.

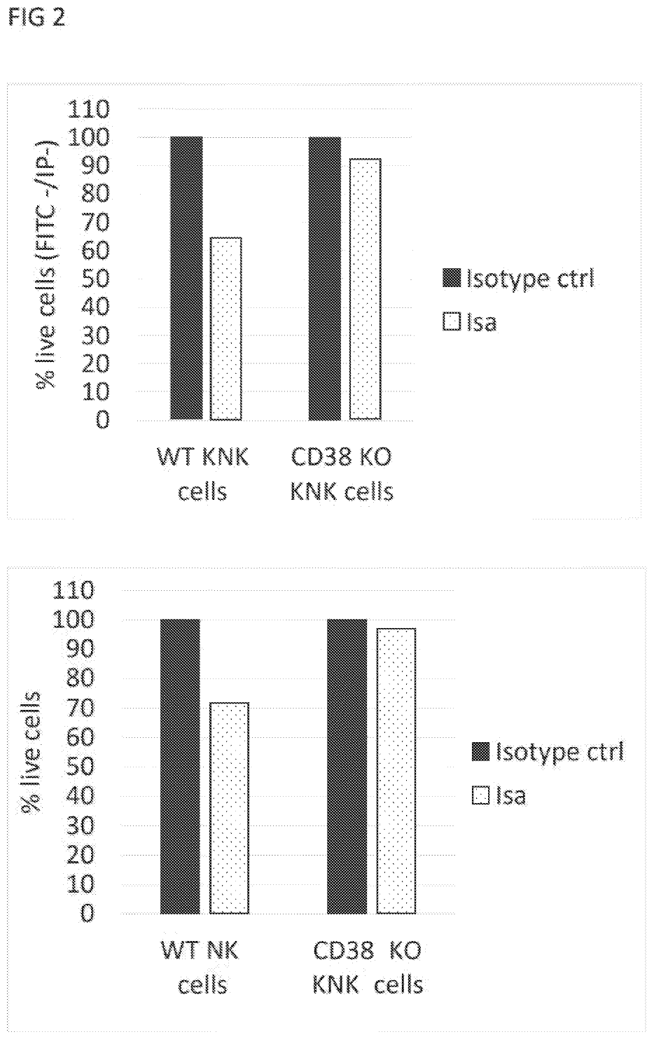

For the graphic representation, viable K-NK cells (DiOC6pos/DRAQ7neg) were quantified as percentage of viable cells in the presence of isatuximab normalized to the percentage of viable cells in the presence of isotype control (100%) (FIG. 2).

Experiments were performed with WT and CD38KO K-NK cells from two donors (BC45 and BC50). As shown in FIG. 1. fratricide effect is avoided with CD38 KO K-NK cells (BC50 having F/V CD16 phenotype) in presence of isatuximab. As shown in FIG. 2. fratricide effect is avoided with CD38 KO K-NK cells (BC45 having V/V CD16 phenotype) in presence of isatuximab. Furthermore, the presence of SAR444245 enhanced the cytotoxic activity of isatuximab against LP-1 cells in the presence of wild type NK cells, and enhanced the cytotoxic activity of isatuximab was even further enhanced in the presence of CD38 KO K-NK cells. The cytotoxic activity of the CD38 KO K-NK cells was sustained over time, up to 90 hours.

Each embodiment herein described may be combined with any other embodiment or embodiments unless clearly indicated to the contrary. In particular, any feature or embodiment indicated as being preferred or advantageous may be combined with any other feature or features or embodiment or embodiments indicated as being preferred or advantageous, unless clearly indicated to the contrary.

All references cited in this application are expressly incorporated by reference herein.

1. Use of an anti-CD38 antibody for the treatment of multiple myeloma, wherein the anti-CD38 antibody is administered to an individual in combination with an Interleukin-2 analog (IL-2 analog), wherein the anti-CD38 antibody is administered on Days 1, 8, 15, and 22 of a first 28-day cycle and then administered on Days 1 and 15 of a 28-day cycle for at least one additional cycle and
administering the IL-2 analog to the individual, wherein the IL-2 analog is administered once every two weeks or once every three weeks.

2. The use according to claim 1, wherein the anti-CD38 antibody is isatuximab and is administered intravenously at a dose of 10 mg/kg.

3. The use according to claim 1, wherein the CD-38 antibody is isatuximab and is administered subcutaneously at a dose of 1400 mg.

4. The use according to any one of claims 1-3, wherein the IL-2 analog comprises a non-natural amino acid, N6-((2-azidoethoxy)-carbonyl)-L-lysine (AzK), and wherein the AzK is pegylated.

5. The use according to claim 1, wherein the IL-2 analog is administered at a dose of 16 ug/kg, 24 ug/kg, or 32 ug/kg.

6. The use according to any one of claims 1-3, or 5, wherein the anti-CD38 antibody is administered on Days 1 and 15 of a 28-day cycle for at least 11 cycles and then administered on Day 1 of a 28-day cycle for one or more additional cycles.

7. The use according to any one of claims 1-3, or 5, wherein the individual received one or more lines of therapy for the treatment of multiple myeloma prior to receiving the first cycle of treatment, wherein the one or more prior lines of therapy include an anti-CD38 medication and an anti-BCMA medication.

8. Use of an anti-CD38 antibody for the treatment of multiple myeloma, wherein the anti-CD38 antibody is administered to an individual in combination with an Interleukin-2 analog (IL-2 analog),
wherein the anti-CD38 antibody is isatuximab and wherein the IL-2 analog comprises a non-natural amino acid, N6-((2-azidoethoxy)-carbonyl)-L-lysine (AzK), wherein the AzK is pegylated,
wherein the isatuximab is administered on Days 1, 8, 15, and 22 of a first 28-day cycle and then administered on Days 1 and 15 of a 28-day cycle for at least one additional cycle,
wherein the isatuximab is administered intravenously at a dose of 10 mg/kg or is administered subcutaneously at a dose of 1400 mg,
wherein the IL-2 analog is administered to the individual once every two weeks or once every three weeks, at a dose of 16 ug/kg, 24 ug/kg, or 32 ug/kg, and
wherein the individual received one or more lines of therapy for the treatment of multiple myeloma prior to receiving the first cycle of treatment, wherein the one or more prior lines of therapy include an anti-CD38 medication and an anti-BCMA medication.

9. The use according to any one of claims 1-3, 5, or 8, wherein Natural Killer (NK) cells having expression of CD38 reduced or knocked-out (NK-CD38KO cells) are also administered to the individual.

10. The use according to claim 9, wherein the NK cells were isolated from a human sample.

11. A method of treating a human individual having multiple myeloma, comprising:
administering an anti-CD38 antibody and an Interleukin-2 analog (IL-2 analog) to the individual, wherein the anti-CD38 antibody is administered on Days 1, 8, 15, and 22 of a first 28-day cycle and then administered on Days 1 and 15 of a 28-day cycle for at least one additional cycle and
administering the IL-2 analog to the individual, wherein the IL-2 analog is administered once every two weeks or once every three weeks.

12. The method according to claim 11, wherein the anti-CD38 antibody is isatuximab and is administered intravenously at a dose of 10 mg/kg.

13. The method according to claim 11, wherein the CD-38 antibody is isatuximab and is administered subcutaneously at a dose of 1400 mg.

14. The method according to any one of claims 11-13, wherein the IL-2 analog comprises a non-natural amino acid N6-((2-azidoethoxy)-carbonyl)-L-lysine (AzK), and wherein the AzK is pegylated.

15. The method of claim 14, wherein the IL-2 analog is administered at a dose of 16 ug/kg, 24 ug/kg, or 32 ug/kg.

16. The method according to any one of claims 11-13, or 15, wherein, the anti-CD38 antibody is administered on Days 1 and 15 of a 28-day cycle for at least 11 cycles followed by administering the anti-CD38 antibody on Day 1 of a 28-day cycle for one or more additional cycles.

17. A method of treating a human individual having multiple myeloma, comprising:
administering an anti-CD38 antibody and an Interleukin-2 analog (IL-2 analog) to the individual,
wherein the anti-CD38 antibody is isatuximab and wherein the IL-2 analog comprises a non-natural amino acid, N6-((2-azidoethoxy)-carbonyl)-L-lysine (AzK), wherein the AzK is pegylated,
wherein the isatuximab is administered on Days 1, 8, 15, and 22 of a first 28-day cycle and then administered on Days 1 and 15 of a 28-day cycle for at least one additional cycle and
wherein the isatuximab is administered intravenously at a dose of 10 mg/kg or is administered subcutaneously at a dose of 1400 mg,
wherein the IL-2 analog is administered to the individual once every two weeks or once every three weeks at a dose of 16 ug/kg, 24 ug/kg, or 32 ug/kg, and
wherein the individual received one or more lines of therapy for the treatment of multiple myeloma prior to receiving the first cycle of treatment, wherein the one or more prior lines of therapy include an anti-CD38 medication and an anti-BCMA medication.

18. The method according to any one of claims 11-13, 15 or 17, wherein Natural Killer (NK) cells having expression of CD38 reduced or knocked-out (NK-CD38KO cells) are also administered to the individual.

19. The method according to claim 18, wherein the NK cells were isolated from a human sample.
